(19) United States
(12) Patent Application Publication
(10) Pub. No.: US 2026/0215969 A1
(43) Pub. Date: Jul. 30, 2026

(54) ROBOTIC SYSTEM FOR PERFORMING CATARACT SURGERY

(71) Applicant: Alcon Inc., Fribourg (CH)

(72) Inventor: Steven T. CHARLES, Memphis, TN (US)

(21) Appl. No.: 19/455,371

(22) Filed: Jan. 21, 2026

Related U.S. Application Data
(60) Provisional application No. 63/749,219, filed on Jan. 24, 2025.

(51) Int. Cl. A61F 9/008; A61B 34/30; A61F 9/00; G06T 5/50; G06T 7/00; G06T 7/50; G06T 7/70

(52) U.S. Cl. CPC A61F 9/008; A61B 34/30; A61F 9/0017; G06T 5/50; G06T 7/0012; G06T 7/50; G06T 7/70; A61F 2009/0087; A61F 2009/00887; G06T 2200/04; G06T 2207/10012; G06T 2207/10101; G06T 2207/20221; G06T 2207/30041

(57) ABSTRACT

In certain embodiments, a robotic system for performing cataract surgery includes a base, a robotic mechanism, and a control system. The robotic mechanism includes a patient interface configured to be coupled to the head of a patient, an end effector configured to receive an exchangeable tool, and sensors that include a polarization camera, a stereo camera, and an optical coherence tomography (OCT) sensor. The control system includes a processor configured to fuse the polarized image data, the stereo image data, and the depth image data to generate fused sensor data of a region of the eye including the cornea and the lens, determine the location of a phacoemulsification tool coupled to the end effector based on the fused sensor data, and control the end effector and the phacoemulsification tool, based on the phacoemulsification tool location, to remove the lens through an incision in the cornea.

### CROSS-REFERENCE TO RELATED APPLICATIONS

This application claims priority to and benefit of U.S. Provisional Patent Application No. 63/749,219, filed January 24, 2025, which is incorporated by reference herein in its entirety, and is hereby expressly made a part of this specification.

### BACKGROUND

The present disclosure relates to a robotic system, and, more particularly, to a robotic system for performing cataract surgery.

The human eye receives light through a clear outer portion called the cornea and focuses the resulting image by way of an ocular crystalline lens onto the retina. The quality of the focused image depends on many factors including the size and shape of the eye, and the transparency of the cornea and lens. When age or disease causes the lens to become less transparent, vision deteriorates because of the diminished image that is transmitted to the retina. This deficiency in the lens of the eye is medically known as a cataract. In addition, the crystalline lens may lose accommodation skills with age, which is called presbyopia. An accepted treatment for these conditions is the surgical removal of the crystalline lens followed by a replacement by an artificial intraocular lens (IOL).

A phacoemulsification tool typically uses ultrasonic energy to remove a cataract. After the opening incision and anterior capsulotomy, the phacoemulsification tool emulsifies the hard nucleus, enabling the ophthalmic surgeon to remove the lens material using suction. A posterior chamber IOL is then inserted into the capsular bag through the incision. The structures of the eye are extremely small and delicate, so cataract surgeries demand the development of exacting and precise skills by ophthalmic surgeons. Replacing a skilled ophthalmic surgeon with an automated system presents many technical challenges.

### SUMMARY

In certain embodiments, a robotic system for performing cataract surgery includes a base, a robotic mechanism coupled to the base, and a control system coupled to the robotic mechanism. The robotic mechanism includes a patient interface configured to be coupled to a head of a patient, an end effector configured to receive an exchangeable tool, and sensors that include a polarization camera configured to generate polarized image data of an eye of the patient, a stereo camera configured to generate stereo image data of the eye of the patient, and an optical coherence tomography (OCT) sensor configured to generate depth image data of the eye of the patient. The control system includes a memory and a processor configured to fuse the polarized image data, the stereo image data, and the depth image data to generate fused sensor data of a region of the eye comprising a cornea and a lens, determine, based on the fused sensor data, a location of a phacoemulsification tool coupled to the end effector of the robotic mechanism, and control, based on the phacoemulsification tool location, the end effector and the phacoemulsification tool to remove the lens through an incision in the cornea.

### BRIEF DESCRIPTION OF THE DRAWINGS

FIG. 1 depicts a perspective view of a robotic system for performing cataract surgery, in accordance with embodiments of the present disclosure.

FIG. 2 depicts a block diagram of the robotic system for performing cataract surgery, in accordance with embodiments of the present disclosure.

FIG. 3 depicts a plan view of an eye of a patient, in accordance with embodiments of the present disclosure.

FIG. 4 depicts a data flow diagram for the robotic system for performing cataract surgery, in accordance with embodiments of the present disclosure.

FIG. 5 depicts a data flow diagram for a data fusion module for the robotic system for performing cataract surgery, in accordance with embodiments of the present disclosure.

### DETAILED DESCRIPTION

Embodiments of the present disclosure advantageously provide a robotic system for performing cataract surgery. The robotic system includes a robotic mechanism and a control system. The robotic mechanism includes multiple sensors, an end effector for exchangeable tools, and a patient interface that are coupled to a common, mechanical frame to provide intrinsic sensor calibration. The control system fuses the data generated by the sensors to provide a robust, precise, three-dimensional image data of the eye of the patient, and determines the location of the exchangeable tools relative to the eye during the cataract procedure based on the fused sensor data.

In certain embodiments, the sensors may include, inter alia, a polarization camera configured to generate polarized image data of the eye of the patient, a stereo camera configured to generate stereo image data of the eye of the patient, and an OCT sensor configured to generate depth image data of the eye of the patient. The polarized image data provide excellent discrimination of transparent or near-transparent lens material, and multi-sensor data fusion that incorporates polarization imaging, stereo vision and OCT is far more robust than single sensor or tracking-based approaches.

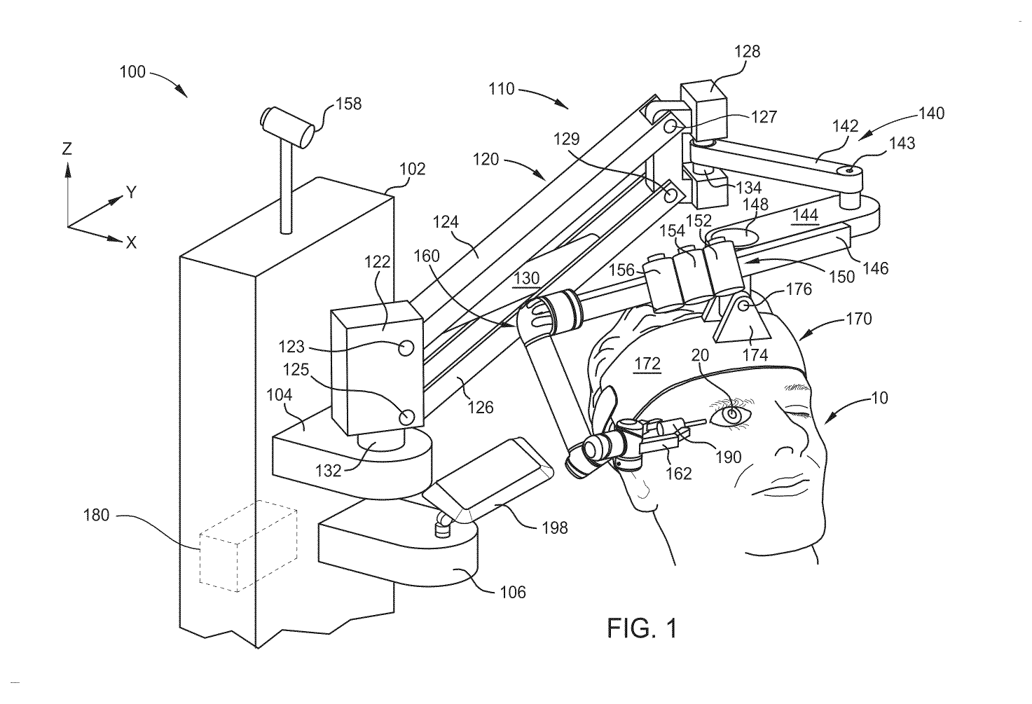

FIG. 1 depicts a perspective view of a robotic system 100 for performing cataract surgery, in accordance with embodiments of the present disclosure.

In certain embodiments, robotic system 100 includes a base 102, a robotic mechanism 110 coupled to the base 102, and a control system 180. The robotic system 100 is located proximate to a surgical table on which the patient is reposed. The base 102 includes a support 104 for the robotic mechanism 110, and a support 106 for a tool holder 198. One or more observation cameras 158 are attached to the base 102 to provide a view of the robotic mechanism 110, the tool holder 198, and the head 10 of the patient. In certain embodiments, the observation cameras 158 may include stereo observation cameras.

The robotic mechanism 110 includes an articulating mechanism 120 coupled to the base 102, a linkage 140 coupled to the articulating mechanism 120, and a patient interface 170 coupled to the linkage 140. In certain embodiments, the articulating mechanism 120 is configured to translate in a first direction, and the linkage 140 is configured to translate in a second direction that is perpendicular to the first direction. For example, the articulating mechanism 120 may translate in a direction that is parallel to a vertical plane (such as the Y-Z plane, the X-Z plane, etc.), and the linkage 140 may translate in a direction that is parallel to a horizontal plane (such as the X-Y plane, etc.), as depicted in FIG. 1.

The articulating mechanism 120 includes a base 122, an upper segment 124, a lower segment 126, an end segment 128, and a spring mechanism 130 coupled to the upper segment 124 and the lower segment 126. Generally, the spring mechanism 130 provides a counter-balancing force for the robotic mechanism 110, and may include one or more compression springs, tension springs, torsion springs, etc.

The base 122 is coupled to the support 104 using a rotary coupling 132, such as rotation about a vertical axis (such as the Z axis, etc.). The upper segment 124 is coupled to the base 122 using a revolute joint 123 (such as a hinge joint, a pin joint, etc.), and to the end segment 128 using a revolute joint 127 (such as a hinge joint, a pin joint, etc.). Similarly, the lower segment 126 is coupled to the base 122 using a revolute joint 125 (such as a hinge joint, a pin joint, etc.), and to the end segment 128 using a revolute joint 129 (such as a hinge joint, a pin joint, etc.). The upper segment 124 is the same length as the lower segment 126.

Due to the lengths of the upper segment 124 and the lower segment 126, the vertical alignment of the revolute joints 123, 125, and the vertical alignment of the revolute joints 127, 129, the end segment 128 translates in a vertical plane (such as the Y-Z plane, etc.).

The linkage 140 includes an upper link 142, a lower link 144, and a mounting rail 146 attached to the lower link 144. The upper link 142 is coupled to the end segment 128 using a revolute joint 134 (such as a hinge joint, a pin joint, etc.), and to the lower link 144 using a revolute joint 143 (such as a hinge joint, a pin joint, etc.). The lower link 144 is also coupled to the patient interface 170 using a revolute joint 148 (such as a hinge joint, a pin joint, etc.).

Sensors 150 are attached to the mounting rail 146, and may include a polarization camera 152, a stereo camera 154, and an optical coherence tomography (OCT) sensor 156. The polarization camera 152 may be configured to generate polarized image data of the eye 20 of the patient, the stereo camera 154 may be configured to generate stereo image data of the eye 20 of the patient, and the OCT sensor 156 may be configured to generate depth image data of the eye 20 of the patient. The end effector 160 is attached to a distal end of the mounting rail 146, and is configured to receive and manipulate exchangeable tools 190 during cataract surgery.

The upper link 142 may have a different length than the lower link 144. The revolute joints 134, 143, 148 are arranged such that their respective axes of rotation are parallel to the vertical plane. Due to the parallel alignment of the revolute joints 134, 143, 148, the lower link 144, the mounting rail 146, and the patient interface 170 translate in a horizontal plane (such as the X-Y plane, etc.).

The articulating mechanism 120 and the linkage 140 may also be locked in a desired orientation to prevent the vertical and horizontal movement of the patient interface 170, as well as the head 10 of the patient, during cataract surgery. For example, the revolute joints 123, 125, 127, 129, 134, 143, 148 may each includes a friction lock, a lockable ratcheting mechanism, etc.

The patient interface 170 includes an adjustable forehead support 172, and a support base 174 attached to the adjustable forehead support 172. The support base 174 is coupled to the lower link 144 using a revolute joint 176 (such as a hinge joint, a pin joint, etc.). The revolute joint 176 is arranged such that its axes of rotation is parallel to the horizontal plane. The adjustable forehead support 172 is attached to the head 10 of the patient during cataract surgery to advantageously maintain the relative distance and orientation of the sensors 150 and the base of the end effector 160 to the eye 20 of the patient, as well as to provide increased patient safety in the context of patient head movement.

In certain embodiments, the adjustable forehead support 172 may be a sterilizable or disposable U-shaped support configured to contact and rest on the forehead and temples of the head 10 of the patient. An underside or patient-facing side of the adjustable forehead support 172 may include a surface padded with, for example, a viscoelastic material, such as dense memory foam, to improve patient comfort. In certain embodiments, the adjustable forehead support 172 may be attached to the head 10 of the patient utilizing a broad and adjustable head strap, which may be fastened via any suitable fastening mechanism. In some embodiments, the head strap may be adjustably fastened via a hook and loop fastener such as, for example, Velcro®, to enable a customized fit with respect to the patient. By attaching the patient interface 170 to the head 10 of the patient via the adjustable forehead support 172, patient head movement relative to the sensors 150 and the base of the end effector 160 is virtually eliminated.

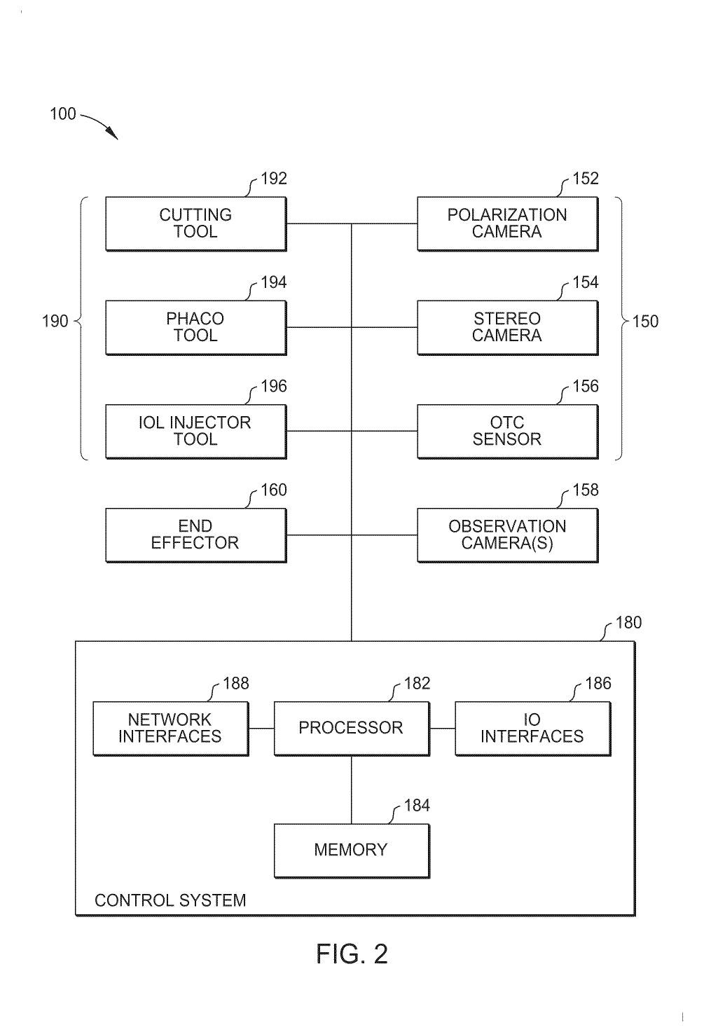

The tool holder 198 is configured to receive and secure exchangeable tools 190 for the robotic mechanism 110, such as a cutting tool 192, a phacoemulsification tool 194, an IOL injector tool 196, etc. (see FIG. 2). Traditionally, an exchangeable tool 190 includes a handpiece that may be gripped by an ophthalmic surgeon. In certain embodiments, the handpiece may be gripped by the end effector 160 of the robotic mechanism 110, such as the fingers 162 of the end effector 160. In some embodiments, the handpiece may be specifically configured to be gripped by the end effector 160 of the robotic mechanism 110, which may be a mechanical gripper, a vacuum gripper, a magnetic gripper, etc.

Each exchangeable tool 190 is coupled to the equipment needed to support the operation of the exchangeable tool 190, such as an electrical power supply, an irrigation fluid pump, an aspiration (vacuum) pump, a femtosecond laser light source, etc. The exchangeable tools 190 are coupled to the support equipment using electrical cables, fluid tubing, fiber optic cables, etc. Additionally, the control system 180 may be coupled to the exchangeable tools 190 using cables that include one or more electrical signal conductors, either directly or via the support equipment.

FIG. 2 depicts a block diagram of the robotic system 100, in accordance with embodiments of the present disclosure.

The control system 180 is communicatively coupled to the sensors 150, the observation camera 158, the end effector 160, and the exchangeable tools 190.

In certain embodiments, the sensors 150 may include a polarization camera 152, a stereo camera 154, and an OCT sensor 156 (as discussed above). In some embodiments, the sensors 150 may also include one or more observation sensors that generate image or other data for use by the control system 180, such as an observation camera, an infrared sensor, an ultrasonic sensor, a light detection and ranging (LIDAR) sensor, etc. The observation sensor data depict at least a portion of the head 10 of the patient (including the eye 20), the end effector 160, the exchangeable tool 190, and the tool holder 198.

In certain embodiments, the end effector 160 may receive commands from the control system 180, such as a commanded rotational displacement for each joint, a commanded position of each finger 162, a commanded force to be applied by each finger 162, etc. Similarly, the end effector 160 may send data to the control system 180, such as the current rotational displacement for each joint, the position of each finger 162, the force applied by each finger 162, etc.

In certain embodiments, the exchangeable tools 190 may include a cutting tool 192, a phacoemulsification tool 194, and an IOL injector tool 196 (as discussed above). In some embodiments, the cutting tool 192 may include a head with a fixed or articulated blade, while in other embodiments, the cutting tool 192 may include a femtosecond laser head. The phacoemulsification tool 194 may include an ultrasonic probe with a tip electrically coupled to an ultrasonic signal source, an irrigation port fluidically coupled to an irrigation source, and an aspiration port fluidically coupled to a suction source. The IOL injector tool 196 may include an articulated IOL injector or plunger mechanism that is configured to insert an IOL into the eye 20 of the patient, such as a monofocal IOL, a toric IOL, a multifocal IOL, an extended depth-of-focus (EDOF) IOL, etc.

In some embodiments, the exchangeable tools 190 may include additional tools for use during cataract surgery, such as an irrigation tool, an aspiration tool, a femtosecond laser tool, etc. Generally, a femtosecond laser may be used as the cutting tool 192 to perform corneal incisions, as a tool to perform capsulorhexis, and as a tool to initially fragment the lens prior to phacoemulsification.

In certain embodiments, the control system 180 includes a processor 182 coupled to a memory 184, I/O interfaces 186, and network interfaces 188.

The I/O interfaces 186 are coupled to the sensors 150, the observation camera 158, the end effector 160, and the exchangeable tools 190. The I/O interfaces 186 may include wired or wireless serial or parallel communication interfaces (such as USB, Bluetooth, etc.) that are configured to receive data from the sensors 150 and the observation camera 158, and send commands to the end effector 160 and the exchangeable tools 190. Similarly, the network interfaces 188 may include wired or wireless network communication interfaces (such as Bluetooth, WiFi, Ethernet, etc.) that may be coupled to one or more wired or wireless networks, such as a local area network (LAN), etc. The network interfaces 188 may communicate with one or more local or remote computer systems. For example, sensor data may be transmitted from the control system 180 to a local computer for presentation to an attending nurse or surgical technician, to a remote computer for presentation to an ophthalmic surgeon monitoring the cataract procedure, etc.

Generally, the processor 182 is configured to execute a method for performing cataract surgery that includes, inter alia, processing the data received from the sensors 150, determining the location of the exchangeable tool 190 coupled to the end effector 160, and controlling the end effector 160 and the exchangeable tool 190 during the cataract procedure. An overview of the cataract procedure will now be described.

At the beginning of the procedure, the end effector 160 is disposed in a stowed position, and all of the exchangeable tools 190 are secured to the tool holder 198.

After the patient is reposed on the surgical table, the adjustable forehead support 172 of the patient interface 170 is attached to the head 10 of the patient by an attending nurse or surgical technician. Because the robotic mechanism 110 is counter-balanced and compliant, the attending nurse may maneuver the robotic mechanism 110 into the proper position. After the adjustable forehead support 172 is attached to the head 10 of the patient, the robotic mechanism 110 may be locked in place or allowed to move with the patient while platform remains parallel to surgical table and floor. At this time, a speculum may be attached to the eye 20, medications may be administered (such as anesthetic eye drops, etc.), etc.

Once the patient has been prepared, the ophthalmic surgeon or the attending nurse may initiate the cataract procedure by sending a start command from the remote or local computer to the control system 180. At any time during the cataract procedure, the ophthalmic surgeon or the attending nurse may abort the cataract procedure by sending an abort command from the remote or local computer to the control system 180, which causes the processor 182 to remove any exchangeable tool 190 that may be located within the eye 20, secure the exchangeable tool 190 to the tool holder 198, and move the end effector 160 to the stowed position.

After the processor 182 receives the start command, the processor 182 is configured to process the observation image data and control the end effector 160 to move from the stowed position to the tool holder 198, couple the cutting tool 192 to the end effector 160 (such as gripping the cutting tool 192 using the fingers 162), and move the cutting tool 192 to a ready position proximate to the eye 20 of the patient (as depicted in FIG. 1). The observation image data may include image data from the observation camera 158 as well as image data from one or more observation sensors attached to the mounting rail 146.

In certain embodiments, the processor 182 may process the observation image data to identify and determine the initial positions of the end effector 160, the tool holder 198, the cutting tool 192, and the eye 20. The processor 182 then determines the commands to control the end effector 160 to move from the stowed position to the tool holder 198. During this time, the processor 182 may employ one or more object tracking methods, including the use of ML models, deep-learning ML models, etc., to track the movement of the end effector 160, which includes predicting the position of the end effector 160 with respect to the locations of the tool holder 198 and the cutting tool 192.

In some embodiments, the processor 182 may model the appearance of the end effector 160 to compensate for distortions of the images of the end effector 160 during movement. The processor 182 may also estimate the motion of the end effector 160 to predict the future position of the end effector 160, and the actual location of the end effector 160 may be determined after the movement has been completed. Generally, this process is repeated for each movement of the end effector 160, and the particular exchangeable tool 190 that is coupled to the end effector 160, during the procedure. In other words, after the exchangeable tool 190 has been coupled to the end effector 160, the processor 182 also tracks the movement of the exchangeable tool 190 during the procedure, as discussed in more detail below.

After the end effector 160 arrives at the tool holder 198, the processor 182 then determines the commands to control the end effector 160 to couple the cutting tool 192 to the end effector 160 (such as gripping the cutting tool 192 using the fingers 162). The processor 182 then determines the commands to control the end effector 160 to move the cutting tool 192 to a ready position proximate to the eye 20 of the patient (as depicted in FIG. 1).

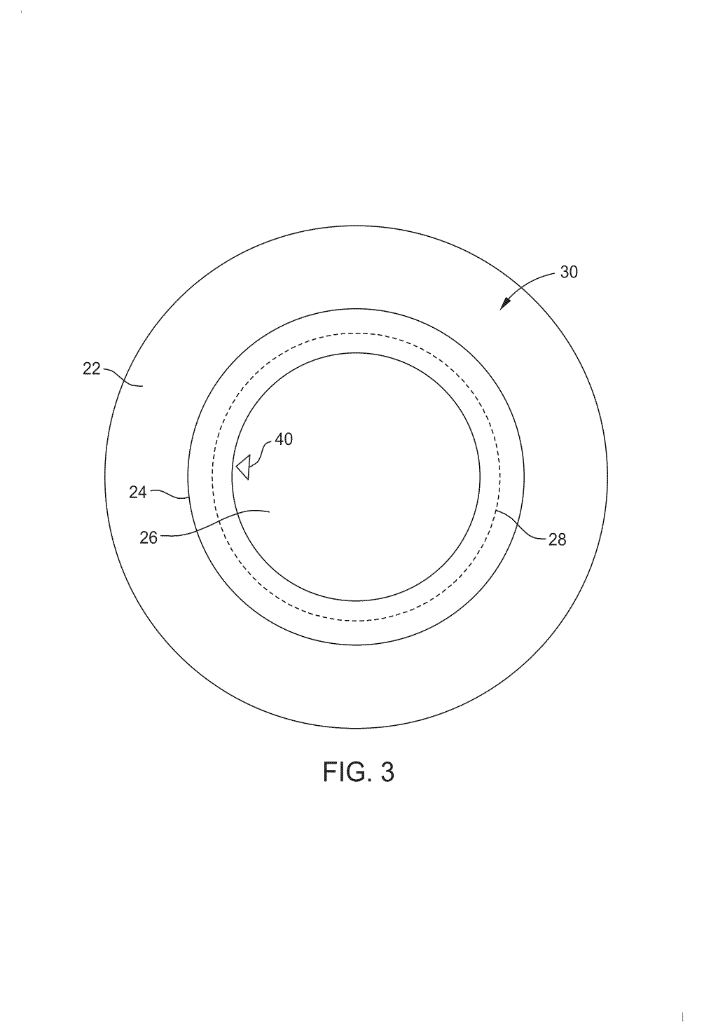

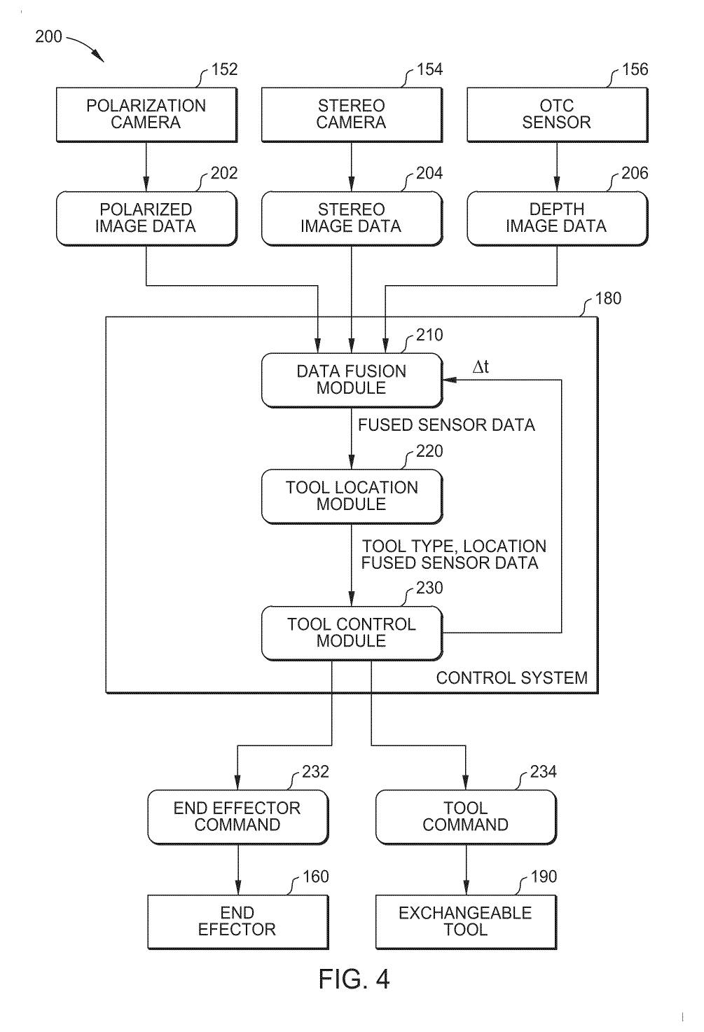

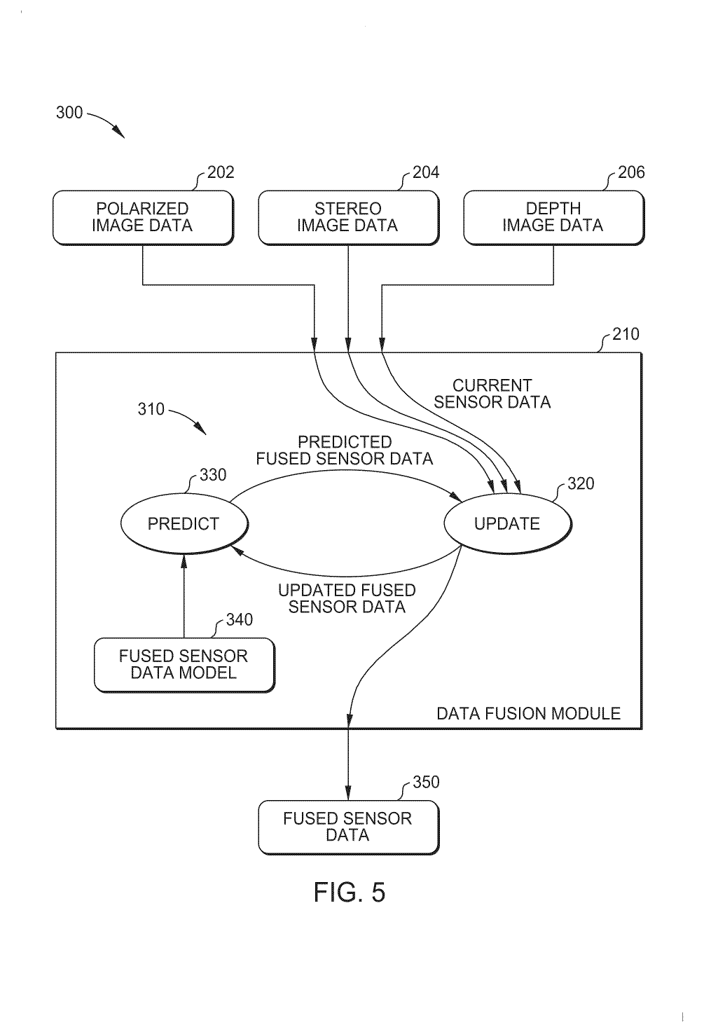

After the cutting tool 192 has been moved to the ready position, the processor 182 is configured to fuse the polarized image data, the stereo image data, and the depth image data to generate fused sensor data of a region of the eye 20 that includes the cornea and the lens. Referring to FIG. 3, the sclera 22, the limbus 24, the cornea 26, and the lens 28 of the eye 20 are depicted, and the region 30 that includes the cornea 26 and the lens 28 is also indicated. The processor 182 is then configured to determine a location of the cutting tool 192 based on the fused sensor data, and control the end effector 160 and the cutting tool 192 to create an incision 40 (FIG. 3) in the cornea of the eye 20 based on the location of the cutting tool 192, as described with respect to FIG. 4. The fused sensor data is periodically generated using a real time data combining process that generates three-dimensional image data, such as a Kalman filter described with respect to FIG. 5.

After the incision is created, the processor 182 is configured to move the cutting tool 192 back to the ready position based on the fused sensor data. The processor 182 is then configured to process the observation image data and control the end effector 160 to move from the ready position to the tool holder 198, secure the cutting tool 192 to the tool holder 198 (thereby decoupling the cutting tool 192 from the end effector 160), couple the phacoemulsification tool 194 to the end effector 160 (such as gripping the phacoemulsification tool 194 using the fingers 162), and move the phacoemulsification tool 194 to the ready position.

After the phacoemulsification tool 194 has been moved to the ready position, the processor 182 is configured to fuse the polarized image data, the stereo image data, and the depth image data to generate fused sensor data of the region 30 of the eye 20, determine a location of the phacoemulsification tool 194 based on the fused sensor data, and control the end effector 160 and the phacoemulsification tool 194 to remove the lens 28 through the incision 40 in the cornea 26 based on the location of the phacoemulsification tool 194.

After the lens is removed, the processor 182 is configured to move the phacoemulsification tool 194 back to the ready position based on the fused sensor data. The processor 182 is then configured to process the observation image data and control the end effector 160 to move the phacoemulsification tool 194 from the ready position to the tool holder 198, secure the phacoemulsification tool 194 to the tool holder 198 (thereby decoupling the phacoemulsification tool 194 from the end effector 160), couple the IOL injector tool 196 to the end effector 160 (such as gripping the IOL injector tool 196 using the fingers 162), and move the IOL injector tool 196 to the ready position.

After the IOL injector tool 196 has been moved to the ready position, the processor 182 is configured to fuse the polarized image data, the stereo image data, and the depth image data to generate fused sensor data of the region 30 of the eye 20, determine a location of the IOL injector tool 196 based on the fused sensor data, and control the end effector 160 and the IOL injector tool 196 to insert an IOL through the incision 40 in the cornea 26 based on the location of the IOL injector tool 196.

After the IOL is inserted, the processor 182 is configured to move the IOL injector tool 196 to the ready position based on the fused sensor data. The processor 182 is then configured to process the observation image data and control the end effector 160 to move from the ready position to the tool holder 198, secure the IOL injector tool 196 to the tool holder 198 (thereby decoupling the IOL injector tool 196 from the end effector 160), and move the end effector 160 to the stowed position.

FIG. 4 depicts a data flow diagram 200 for the robotic system 100, in accordance with embodiments of the present disclosure.

Generally, certain functionality described above may be embodied within one or more software modules that are stored in the memory 184 and executed by the processor 182. In certain embodiments, the software modules may include, inter alia, a data fusion module 210, a tool location module 220, and a tool control module 230. Polarized image data 202 are generated by the polarization camera 152, and periodically provided to the data fusion module 210. Stereo image data 204 are generated by the stereo camera 154 and periodically provided to the data fusion module 210. Depth image data 206 are generated by the OCT sensor 156, and periodically provided to the data fusion module 210.

The data fusion module 210 periodically generates fused sensor data of the region 30 based on the polarized image data 202, the stereo image data 204, and the depth image data 206. More particularly, the data fusion module 210 uses a data combining process that generates three-dimensional image data of the region 30, such as a Kalman filter, etc. In other words, the fused sensor data provide a three-dimensional map of the structures and cavities of the eye 20, such as the cornea 26, the lens 28, the anterior chamber of the anterior cavity, the posterior chamber of the anterior cavity, etc., as well as the portion of an exchangeable tool 190 that may be present within the region 30. The data fusion module 210 periodically provides the fused sensor data to the tool location module 220 at predetermined time intervals tᵢ that are separated by Δt seconds.

The tool location module 220 periodically processes the fused sensor data to recognize the portion of an exchangeable tool 190 that may be present within the region 30 (such as the tip), and then determines the location of the tip of the exchangeable tool 190 within the region 30. For example, the tool location module 220 may include an image recognition module, such as an ML model, etc., that is configured to identify the tip of each exchangeable tool 190 and determine the location of the exchangeable tool 190 within the region 30. The tool location module 220 periodically provides the type and location of the exchangeable tool 190, as well as the fused sensor data, to the tool control module 230. Alternatively, the data fusion module 210 may provide the fused sensor data directly to the tool control module 230.

The tool control module 230 first determines a three-dimensional path for the tip of the exchangeable tool 190 to follow through the three-dimensional image data of the region 30 based on the location of the exchangeable tool 190 and the fused sensor data. The tool control module 230 then determines the appropriate end effector commands 232 for the joints of the end effector 160 to effectuate the movement of the tip of the exchangeable tool 190 along the three-dimensional path, and the appropriate tool commands 234 for the exchangeable tool 190 to perform its function along the path, such as cutting, fragmentation, emulsification, lens removal, IOL insertion, etc. The end effector commands 232 are periodically provided to the end effector 160, and the tool commands 234 are periodically provided to the appropriate exchangeable tool 190.

In other words, the tool control module 230 controls the end effector 160 and the exchangeable tool 190 to perform the exchangeable tool’s function based on the location of the exchangeable tool 190. For example, the tool control module 230 controls the end effector 160 and the cutting tool 192 to create the incision 40 in the cornea 26 of the eye 20 based on the location fo the cutting tool 192. Similarly, the tool control module 230 controls the end effector 160 and the phacoemulsification tool 194 to remove the lens 28 through the incision 40 in the cornea 26 based on the location of the phacoemulsification tool 194. The tool control module 230 also controls the end effector 160 and the IOL injector tool 196 to insert an IOL through the incision 40 in the cornea 26 based on the location of the IOL injector tool 196.

FIG. 5 depicts a data flow diagram 300 for a data fusion module 210, in accordance with embodiments of the present disclosure.

As described above, the data fusion module 210 periodically receives the polarized image data 202, the stereo image data 204, and the depth image data 206, and periodically provides the fused sensor data 350 to the tool location module 220. In some embodiments, the data fusion module 210 may also periodically provide the fused sensor data 350 directly to the tool control module 230.

In certain embodiments, the data fusion module 210 periodically generates fused sensor data 350 of the region 30 based on the polarized image data 202, the stereo image data 204, and the depth image data 206 using a Kalman filter 310. More particularly, the data fusion module 210 executes the Kalman filter 310 at predetermined time intervals tᵢ that are separated by ∆t seconds.

The Kalman filter 310 may include an update module 320, a predict module 330, and a fused sensor data model 340. At each predetermined time interval tᵢ, the Kalman filter 310 generates predicted fused sensor data based on previous fused sensor data and the fused sensor data model 340, and generates the fused sensor data 350 for the predetermined time interval tᵢ based on the predicted fused sensor data, the polarized image data 202, the stereo image data 204, and the depth image data 206.

As discussed above, polarized image data 202 advantageously provides provide excellent discrimination of transparent or near-transparent cataract and lens material after fragmentation, pre-chopping, chopping, cracking, segmentation, etc. Additionally, both the polarized image data 202 and the depth image data 206 advantageously provide time-of-flight (TOF) range information. In some embodiments, the data fusion module 210 may generate disparity maps from the stereo image data 204, which may be used to generate depth maps using epipolar geometry and triangulation methods. Further, multi-sensor data fusion that incorporates polarized image data 202, stereo image data 204, and depth image data 206 is far more robust than single sensor or tracking-based approaches.

Generally, sensor calibration provides the position and orientation of the sensors 150 in a global or “real-world” coordinate system (such as the XYZ roll pitch yaw (6-DOF) coordinate system depicted in FIG. 1) by comparing the relative positions of known features detected by the sensors 150. Precise sensor calibrations are vital for further processing, such as data fusion, obstacle detection, recognition, localization and mapping, end effector 160 and exchangeable tool 190 control, etc. Because the robotic mechanism 110 provides fixed, rigid, known mechanical relationships between the sensors 150 and the articulating mechanism 120, the linkage 140, the base of the end effector 160, and the patient interface 170, the robotic system advantageously provides intrinsic sensor calibration.

The many features and advantages of the disclosure are apparent from the detailed specification, and, thus, it is intended by the appended claims to cover all such features and advantages of the disclosure which fall within the scope of the disclosure. Further, since numerous modifications and variations will readily occur to those skilled in the art, it is not desired to limit the disclosure to the exact construction and operation illustrated and described, and, accordingly, all suitable modifications and equivalents may be resorted to that fall within the scope of the disclosure.

What is claimed is:

1. A robotic system for performing cataract surgery, comprising:
a base;
a robotic mechanism coupled to the base, the robotic mechanism comprising:
a patient interface configured to be coupled to a head of a patient,
an end effector configured to receive an exchangeable tool, and
sensors comprising:
a polarization camera configured to generate polarized image data of an eye of the patient,
a stereo camera configured to generate stereo image data of the eye of the patient, and
an optical coherence tomography (OCT) sensor configured to generate depth image data of the eye of the patient; and
a control system coupled to the robotic mechanism, the control system comprising a memory and a processor configured to:
fuse the polarized image data, the stereo image data, and the depth image data to generate fused sensor data of a region of the eye comprising a cornea and a lens,
determine, based on the fused sensor data, a location of a phacoemulsification tool coupled to the end effector of the robotic mechanism, and
control, based on the phacoemulsification tool location, the end effector and the phacoemulsification tool to remove the lens through an incision in the cornea.

2. The robotic system of claim 1, wherein the fused sensor data comprises three-dimensional image data of the region of the eye.

3. The robotic system of claim 2, wherein the processor is configured to generate the fused sensor data using a data combining process.

4. The robotic system of claim 3, wherein the data combining process comprises a Kalman filter, and the processor is further configured to:
execute the Kalman filter at predetermined time intervals tᵢ that are separated by ∆t milliseconds; and
at each predetermined time interval tᵢ:
generate, based on previous fused sensor data and a model of the fused sensor data, predicted fused sensor data, and
generate, based on the predicted fused sensor data, the polarized image data, the stereo image data, and the depth image data, the fused sensor data for the predetermined time interval tᵢ.

5. The robotic system of claim 2, wherein the processor is further configured to:
determine, based on the fused sensor data, a location of a cutting tool coupled to the end effector of the robotic mechanism; and
control, based on the cutting tool location, the end effector and the cutting tool to create the incision in the cornea of the eye.

6. The robotic system of claim 5, wherein the processor is further configured to:
determine, based on the fused sensor data, a location of an intraocular lens (IOL) injector tool coupled to the end effector of the robotic mechanism; and
control, based on the IOL injector tool location, the end effector and the IOL injector tool to insert an IOL through the incision in the cornea of the eye.

7. The robotic system of claim 6, further comprising:
one or more observation cameras configured to generate observation image data,
wherein the processor is further configured to:
control, based on the observation image data, the end effector to couple the cutting tool to the end effector,
after the incision is created, control, based on the observation image data, the end effector to decouple the cutting tool from the end effector and couple the phacoemulsification tool to the end effector,
after the lens is removed through the incision in the cornea, control, based on the observation image data, the end effector to decouple the phacoemulsification tool from the end effector and couple the IOL injector tool to the end effector, and
after the IOL is inserted through the incision in the cornea, control, based on the observation image data, the end effector to decouple the IOL injector tool from the end effector.

8. The robotic system of claim 1, wherein the robotic mechanism further comprises:
an articulating mechanism coupled to the base, the articulating mechanism configured to translate in a first direction; and
a linkage coupled to the articulating mechanism and the patient interface, the linkage configured to translate in a second direction perpendicular to the first direction,
wherein the articulating mechanism is counter-balanced, and
wherein the end effector and the sensors are attached to the linkage.

9. The robotic system of claim 8, wherein the first direction is parallel to a vertical plane, and the second direction is parallel to a horizontal plane.

10. The robotic system of claim 9, wherein the patient interface comprises an adjustable forehead support.

11. The robotic system of claim 1, wherein the phacoemulsification tool comprises an ultrasonic probe including a tip, an irrigation port, and an aspiration port.

12. The robotic system of claim 5, wherein the cutting tool comprises a femtosecond laser that is further configured to at least partially fragment the lens.

13. The robotic system of claim 6, wherein the IOL comprises a monofocal IOL, a toric IOL, a multifocal IOL, or an extended depth-of-focus (EDOF) IOL.

14. A method for performing cataract surgery using a robotic system, comprising:
generating, by a polarization camera, polarized image data of an eye of a patient;
generating, by a stereo camera, stereo image data of the eye of the patient;
generating, by an optical coherence tomography (OCT) sensor, depth image data of the eye of the patient;
fusing, by a processor, the polarized image data, the stereo image data, and the depth image data to generate fused sensor data of a region of the eye comprising a cornea and a lens;
determining, by the processor based on the fused sensor data, a location of a phacoemulsification tool coupled to an end effector of a robotic mechanism; and
controlling, by the processor based on the phacoemulsification tool location, the end effector and the phacoemulsification tool to remove the lens through an incision in the cornea.

15. The method of claim 14, wherein:
the fused sensor data comprises three-dimensional image data of the region of the eye; and
the fused sensor data is generated using a data combining process.

16. The method of claim 15, wherein the data combining process is a Kalman filter, and generating the fused sensor data comprises:
executing the Kalman filter at predetermined time intervals tᵢ that are separated by ∆t milliseconds; and
at each predetermined time interval tᵢ:
generating, based on previous fused sensor data and a model of the fused sensor data, predicted fused sensor data, and
generating, based on the predicted fused sensor data, the polarized image data, the stereo image data, and the depth image data, the fused sensor data for the predetermined time interval tᵢ.

17. The method of claim 14, further comprising:
determining, by the processor based on the fused sensor data, a location of a cutting tool coupled to the end effector of the robotic mechanism;
controlling, by the processor based on the cutting tool location, the end effector and the cutting tool to create the incision in the cornea of the eye;
determining, by the processor based on the fused sensor data, a location of an intraocular lens (IOL) injector tool coupled to the end effector of the robotic mechanism; and
controlling, by the processor based on the IOL injector tool location, the end effector and the IOL injector tool to insert an IOL through the incision in the cornea of the eye.

18. The method of claim 17, further comprising:
generating, by one or more observation cameras, observation image data;
controlling, by the processor based on the observation image data, the end effector to couple the cutting tool to the end effector,
after the incision is created, controlling, by the processor based on the observation image data, the end effector to decouple the cutting tool from the end effector and couple the phacoemulsification tool to the end effector,
after the lens is removed through the incision in the cornea, controlling, by the processor based on the observation image data, the end effector to decouple the phacoemulsification tool from the end effector and couple the IOL injector tool to the end effector, and
after the IOL is inserted through the incision in the cornea, controlling, by the processor based on the observation image data, the end effector to decouple the IOL injector tool from the end effector.

19. The method of claim 18, wherein:
the phacoemulsification tool comprises an ultrasonic probe including a tip, an irrigation port, and an aspiration port;
the cutting tool comprises a femtosecond laser that is further configured to at least partially fragment the lens; and
the IOL comprises a monofocal IOL, a toric IOL, a multifocal IOL, or an extended depth-of-focus (EDOF) IOL.

20. The method of claim 14, wherein:
the robotic system comprises:
an articulating mechanism coupled to a base, the articulating mechanism configured to translate in a first direction, and
a linkage coupled to the articulating mechanism and a patient interface, the linkage configured to translate in a second direction perpendicular to the first direction,
the articulating mechanism is counter-balanced;
the polarization camera, the stereo camera, the OCT sensor, and the end effector are attached to the linkage;
the first direction is parallel to a vertical plane, and the second direction is parallel to a horizontal plane; and
the patient interface comprises an adjustable forehead support.
